(19) United States
(12) Patent Application Publication
(10) Pub. No.: US 2026/0354806 A1
(43) Pub. Date: Nov. 12, 2026

(54) RELEASABLE COUPLING FOR A MEDICAL DEVICE AND RELATED METHODS

(71) Applicant: Boston Scientific Scimed, Inc., Maple Grove, MN (US)

(72) Inventors: Madeline Anne ROBERTSON, Greenville, SC (US); Lillian SHOEMAKER-CONOVER, Greene, ME (US)

(73) Assignee: Boston Scientific Scimed, Inc., Maple Grove, MN (US)

(21) Appl. No.: 19/669,578

(22) Filed: May 6, 2026

Related U.S. Application Data
(60) Provisional application No. 63/801,223, filed on May 7, 2025.

(51) Int. Cl. A61B 10/04; A61B 10/06; A61B 10/02

(52) U.S. Cl. CPC A61B 10/04; A61B 10/06; A61B 2010/0208

(57) ABSTRACT

Aspects of this disclosure include a medical device having a handle, a shaft extending distally from the handle, an end effector assembly removably coupled to a distal end of the shaft, and a shaft cover fixed to the end effector assembly. The shaft cover is configured to extend proximally over at least a portion of the shaft. In the first configuration of the shaft cover, the shaft cover is at least partially rolled.

### CROSS-REFERENCE TO RELATED APPLICATIONS

This application claims the benefit of priority of U.S. Provisional Patent Application No. 63/801,223, filed on May 7, 2025, the entirety of which is incorporated herein by reference.

### TECHNICAL FIELD

The disclosure relates generally to a releasable coupling for medical devices. More specifically, aspects of the disclosure pertain to a releasable coupling for assembling an end effector to a medical device, such as a removable sheath.

### BACKGROUND

Many endoscopic devices, including for example hemostasis clips and biopsy forceps, are currently single-use devices. Such devices often include a handle, a shaft, and a distal tip that includes an end effector. The distal tip and the shaft are inserted in the body to perform a procedure at a tissue site (e.g., obtain a tissue sample), while the handle remains outside of the body. Following a procedure, an entirety of the medical device, including the handle, shaft, and distal tip, may be disposed of.

### SUMMARY

This disclosure includes devices and methods that allow for releasably coupling a first portion of a medical device (e.g., a handle and a shaft) to a second portion of the medical device (e.g., an end effector and a cover or other portions that are inserted in the body during use). Such devices and methods permit, for example, re-use of the first portion with other second portions, resulting in less waste. Each of the aspects disclosed herein may include one or more of the features described in connection with any of the other disclosed aspects. The examples described herein may have any of these features in any combination.

Aspects of this disclosure include a medical device having a handle, a shaft extending distally from the handle, an end effector assembly removably coupled to a distal end of the shaft, and a shaft cover fixed to the end effector assembly. The shaft cover is configured to extend proximally over at least a portion of the shaft. In a first configuration of the shaft cover, the shaft cover is at least partially rolled.

The medical device may include one or more of the following features alone or in any combination. In a second configuration of the shaft cover, the shaft cover may be unrolled and extend proximally along at least a portion of a longitudinal length of the shaft. In the second configuration, the shaft cover may extend along an entire longitudinal length of the shaft. The shaft cover may include a membrane extending across an opening at a proximal portion of the end effector assembly.

The medical device may further include an actuation element extending proximally from the end effector assembly. The actuation element may extend through the distal membrane.

The medical device may further include a first actuation element extending from the end effector assembly and terminating at a distal side of the membrane, and a second actuation element extending from the handle and terminating at a proximal side of the membrane. The membrane may be disposed between the first actuation element and the second actuation element. A distal end of the second actuation element may be coupled to a fastener disposed on the membrane.

The end effector assembly may include a base portion configured to receive a distal end of the shaft. The base portion may define an opening through which an actuation element extends. The base portion may include one or more protrusions extending radially inward from an internal surface of the base portion. The one or more protrusions may be configured to interact with complementary indentations disposed on an outer surface of the shaft. The base portion may include a slit extending distally from a proximal end of the base portion. The base portion may be configured to at least partially expand at the slit upon receiving the distal end of the shaft.

A proximal end of the shaft cover may be secured to the handle via a clamp configured to surround the shaft cover and a portion of the handle. The handle may include a distal lock configured to secure a proximal end of the shaft cover between a proximal end of the shaft and the distal lock. The end effector assembly may include an end effector having a pair of jaws movably fixed relative to one another, and a pair of arms extending distally from a base portion. The shaft includes a flexible coil.

According to another aspect, a medical device includes a handle, a shaft extending distally from the handle, an end effector assembly removably coupled to a distal end of the shaft, and a shaft cover fixed to the end effector assembly. The end effector assembly includes an end effector and a base portion defining an opening. The shaft cover includes a membrane extending across the opening. The membrane is a barrier to inhibit material from entering a lumen of the shaft.

According to other aspects of the present disclosure, the medical device may include one or more of the following features alone or in any combination. The medical device may further include an actuation element extending proximally from the end effector. The actuation element may extend through the membrane and extend proximally through the shaft to the handle. The medical device may further include a first actuation element extending proximally from the end effector and terminating distally of the membrane, and a second actuation element extending from the handle and terminating proximally of the membrane. The membrane may be disposed between a proximalmost end of the first actuation element and a distalmost end of the second actuation element. The distalmost end of the second actuation element may be secured to a fastener disposed on a surface of the membrane.

According to another aspect of the present disclosure, a distal end effector assembly of a medical device is provided. The distal end effector assembly includes a pair of jaws movably fixed relative to one another, an actuation element extending proximally from a proximal end of the jaws, a base portion, and a shaft cover fixed to the base portion. A proximal end of the base portion is configured to removably receive a distal end of a shaft. The base portion includes an opening through which the actuation element extends. The shaft cover includes a membrane extending across the opening. The membrane is configured as a barrier to prevent bodily fluids, debris, or contaminants from entering a lumen of the shaft.

It may be understood that both the foregoing general description and the following detailed description are exemplary and explanatory only and are not restrictive of the invention, as claimed. As used herein, the terms “comprises,” “comprising,” “has,” “having,” “includes,” “including,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.” The term “distal” refers to a direction away from an operator/toward a treatment site, and the term “proximal” refers to a direction toward an operator. The term “approximately,” or like terms (e.g., “substantially”), includes values +/- 10 % of a stated value. The term “or” is used disjunctively, such that “at least one of A or B” includes, (A), (B), (A and A), (A and B), etc. The term “or” also includes “and/or.”

### BRIEF DESCRIPTION OF THE DRAWINGS

The accompanying drawings, which are incorporated in and constitute a part of this specification, illustrate examples of this disclosure and, together with the description, serve to explain the principles of the disclosure.

FIG. 1 illustrates a perspective view of an exemplary medical device, including an enlarged view of a distal portion of the medical device, according to aspects of this disclosure;

FIGS. 2A-2C illustrate various views of the distal portion of the medical device of FIG. 1, according to aspects of this disclosure;

FIGS. 3A-3D illustrate side views of the distal portion of the medical device of FIG. 1 in various states of assembly, according to aspects of this disclosure;

FIG. 4 illustrates a side view of a proximal portion of a connected assembly of the medical device of FIG. 1, according to aspects of this disclosure;

FIG. 5 illustrates a side view of a proximal portion of an alternative connected assembly of the medical device of FIG. 1, according to aspects of this disclosure.

### DETAILED DESCRIPTION

Reference is now made in detail to examples of this disclosure, aspects of which are illustrated in the accompanying drawings. Wherever possible, the same reference numbers will be used throughout the drawings to refer to the same or like parts. Reference numbers having the same tens and ones digits indicate corresponding elements across different devices, where feasible.

Embodiments of this disclosure include a medical device that has a first portion that releasably couples to a second portion. The first portion may include a handle and a shaft. The second portion may include an end effector, at least one actuation element, and a cover or sheath. The second portion, which contacts the patient during use, may be disposed of, and the first portion may be cleaned as needed and re-used with a second distal portion in a subsequent procedure on a same patient or a different patient. Such embodiments of a medical device may result in reduced waste and reduced overall cost of the medical device (fewer handles needed, less packaging, etc.). Such embodiments also may result in a universal first portion (e.g., including a handle) that may be used with numerous different types of second portions (including, for example, different types of end effectors).

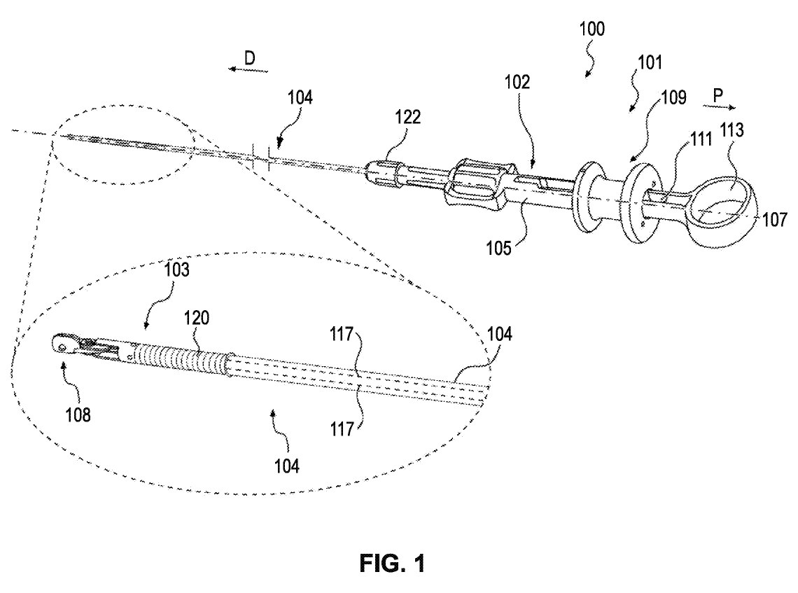

FIG. 1 illustrates a medical device 100 including a first portion 101 and a second portion 103. In aspects, first portion 101 may include a shaft 104 extending distally from a handle 102. First portion 101 may be configured to be single-use or multi-use. Accordingly, first portion 101 may be comprised of materials capable of undergoing sterilization procedures. Second portion 103 may include an end effector 108. Second portion 103 may be removably coupled to first portion 101, such that end effector 108 is removably coupled to a distal end of shaft 104. In some aspects, end effector 108 may include a pair of jaws, for example, configured to extract a sample of tissue from a target site. However, end effector 108 may be any suitable type of instrument (e.g., snare, stent delivery system, basket, balloon, knife, needle, clip, forceps, suture device, etc.). Second portion 103 may be single-use.

Second portion 103 may also include a shaft cover 120, which may be a sheath. Shaft cover 120 extends proximally from end effector 108. Shaft cover 120 may be configured to circumferentially surround shaft 104 and extend proximally along at least a portion of a longitudinal length of shaft 104. In some aspects, shaft cover 120 may extend along an entire longitudinal length of shaft 104 and be secured to handle 102, as will be described in further detail below. In other aspects, shaft cover 120 may extend along only a portion of a longitudinal length of shaft 104 and be secured to shaft 104 distally of handle 102. Portions or an entirety of shaft cover 120 may be configured to conform to a size and shape of handle 102 or shaft 104.

In aspects where shaft cover 120 extends proximally to handle 102 or to a more proximal portion of shaft 104, FIG. 1 may depict shaft cover 120 in a partially applied, or a partially unrolled, state. In other words, for such embodiments, shaft cover 120 is shown in a partially assembled state in FIG. 1. In other aspects, FIG. 1 may depict a fully assembled configuration of medical device 100, in which shaft cover 120 extends along only a distal portion of shaft 104.

Shaft 104 may include one or more lumens configured to receive one or more actuation elements 117 extending proximally from end effector 108. For example, actuation element(s) may be wires, cables, strings, etc., that extend proximally from end effector 108, through shaft 104, and to handle 102. A proximal end of each actuation element 117 may be secured to one or more portions of handle 102. For example, a proximal end of each actuation element 117 may be secured directly or indirectly to one or more actuators 109 of handle 102. Although two actuation elements 117 are shown, medical device 100 may include only one actuation element 117 in some embodiments. Accordingly, actuation element(s) 117 may be movable (e.g., within handle 102 and shaft 104) via manipulation of the one or more actuators 109. In these aspects, manipulation of actuator 109 may control actuation or activation of end effector 108.

In some aspects, shaft 104 may be comprised of a metallic material (e.g., stainless steel, nitinol, etc.), a non-metallic material, such as plastic (e.g., thermoplastic elastomer (TPE) or thermoplastic polyurethane (TPU), or thermoplastic polyesters), polyamide (e.g., nylon or Pebax), polytetrafluoroethylene (PTFE), or any other suitable non-metal (e.g., insulating) material. In some aspects, shaft 104 may be formed as a flexible coil or tube.

A proximal end of shaft 104 may be secured to handle 102. For example, handle 102 may include a body 105 extending along a central longitudinal axis 107 of medical device 100. Body 105 may include one or more inner surfaces defining a lumen or channel of handle 102 which may, for example, be configured to receive or otherwise be coupled (e.g., directly or indirectly) to shaft 104. In some aspects, body 105 may include a distal lock 122 configured to secure shaft cover 120 and shaft to handle 102, as described in further detail below.

Actuator 109 may be a spool having an indented portion (e.g., with a relatively smaller lateral cross-section) and one or more (e.g., two) ridged or extended portions (e.g., flanges with a relatively larger lateral cross-section). In these aspects, the indented portion may receive one or more of a user’s fingers, such that movement of the user’s finger(s) controls the movement of actuator 109. Actuator 109 may be fixedly coupled to actuation element(s) 117 that control end effector 108 or other aspects of distal portion. Although a spool is shown in FIG. 1, actuator 109 may alternatively be another type of actuator (e.g., finger rings, knob, lever, slider, joystick, etc.).

Body 105 may further include a slot 111, for example, extending longitudinally through a portion of body 105. A portion of actuator 109 may extend into a portion of slot 111, such that actuator 109 is movable (e.g., longitudinally movable distally and/or proximally) along slot 111. In these aspects, slot 111 may define a range of movement for actuator 109. Actuator 109 may be movable within slot 111, for example, to control one or more aspects of distal portion, such as to control actuation or activation of end effector 108.

Handle 102 may also include a ring 113 (e.g., thumb ring), for example, at a proximal end of body 105. Ring 113 may accommodate a thumb of a user’s hand when the user engages actuator 109.

In aspects, handle 102 may be formed from stainless steel (e.g., LE sintered stainless steel) or other metallic materials that are suitable for autoclaving or other types of sterilization methods. In other aspects, handle 102 may be formed from plastics or other materials that are suitable for autoclaving or other types of sterilization methods. Handle 102 (e.g., body 105, actuator 109, or finger ring 113) may have colorings or markings indicative of a position or status of medical device 100. For example, handle body 105 may include a first marking and a second marking at or near slot 111 to indicate a first position of end effector 108 (e.g., a closed position) and a second position of end effector 108 (e.g., an open position). Actuator 109 may include a third marking such that, when the third marking of the actuator 109 is aligned with the first marking or the second marking, end effector 108 is in the first position or the second position.

In some aspects, components of second portion 103 (e.g., end effector 108, actuation elements 117, shaft cover 120, etc.) may include disposable and/or recyclable materials, for example to discard after the medical procedure. These components having disposable or recyclable material may be detached from handle 102, separated from other components, and discarded. Handle 102 and shaft 104 may be detached from second portion 103 to, for example, clean and reuse handle 102 and shaft 104.

During use, end effector 108 and at least a distal portion of shaft 104 may be inserted directly into a lumen of a subject or delivered through a working channel or other lumen of an endoscope, duodenoscope, gastroscope, colonoscope, ureteroscope, bronchoscope, or other medical delivery systems to a target site. Thus, medical device 100 may be an endoscopic instrument (e.g., an endoscopic accessory device). Once end effector 108 is in a desired position, actuator 109 may be translated proximally or distally relative to body 105 to actuate or active end effector 108.

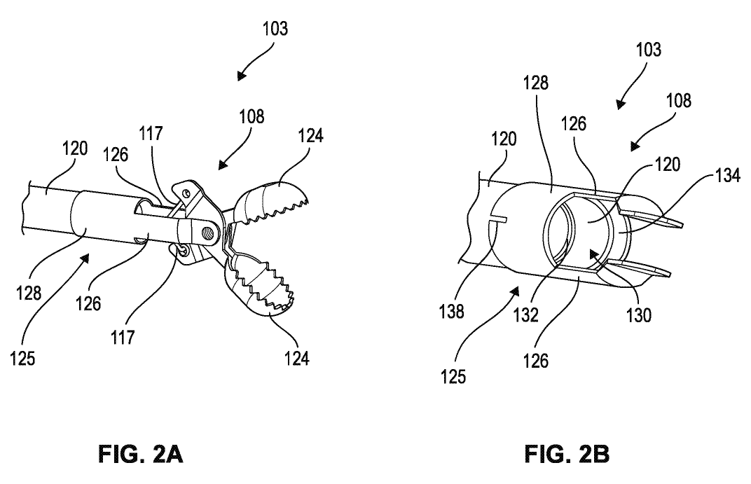

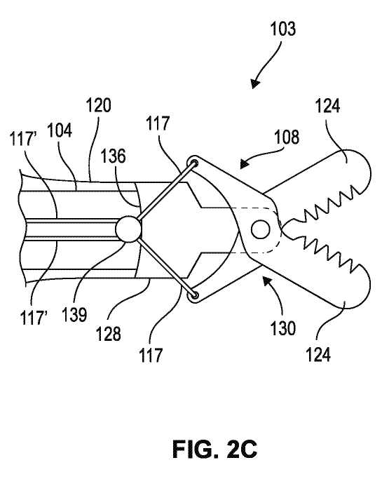

FIG. 2A is a perspective view of end effector 108 removed or disassembled from shaft 104. FIG. 2B is a perspective distal-to-proximal view of end effector 108 with some components removed to better depict the internal surfaces of end effector 108. FIG. 2C is a cross-sectional view of end effector 108.

As previously discussed, end effector 108 may include opposing jaws 124 movably fixed relative to one another. Opposing jaws 124 may be movable relative to one another via one or more actuation elements 117. In such aspects, end effector 108 may include a clevis 125 having a pair of arms 126 extending distally from a base portion 128 of an end effector assembly. Base portion 128 may be configured to receive a distalmost end of shaft 104, as described further below. Accordingly, base portion 128 may define an opening 130 (FIG. 2B), through which actuation element(s) 117 may extend.

Referring to FIG. 2C, in some embodiments, cover 120 includes a distal membrane 136 extending across opening 130. Distal membrane 136 may also be referred to herein as a membrane. Distal membrane 136 may be configured as a barrier to prevent or otherwise inhibit materials such as bodily fluids, debris, or other contaminants from entering the lumen of shaft 104. In some configurations, actuation element(s) 117 penetrate through distal membrane 136 and extend proximally through shaft 104 to handle 102, maintaining a continuous connection therebetween. Alternatively, actuation element(s) 117 may extend from jaws 124 and terminate at the distal side of distal membrane 136, while separate corresponding actuation element(s) 117' may extend from handle 102 to the proximal side of distal membrane 136. A proximal end of each actuation elements 117' may be fixed to the distal side of distal membrane 136 via adhesive bonding, welding, or mechanical fastening techniques suitable for the materials used in the distal membrane and actuation elements. In some aspects, the proximal end of each actuation element 117' may be secured to a fastener, similar to a fastener 139, described below. This arrangement may create a mechanical coupling interface at distal membrane 136, effectively transferring actuation forces between the handle-side actuation elements and the jaw-side actuation elements while maintaining the fluid barrier.

In some aspects, the distal end of the separate actuation element(s) 117' may be fixed directly to the proximal surface of distal membrane 136. This configuration may provide a secure attachment point for transferring actuation forces across the membrane barrier. The direct fixation may involve adhesive bonding, welding, or mechanical fastening techniques suitable for the materials used in the distal membrane and actuation elements. For example, the distal end of the separate actuation element(s) 117' may be secured to fastener 139 disposed on the proximal surface of distal membrane 136. The fastener 139 may be a hook, a welded ball, or other fastener configured to receive a complementary feature at a distal end of separate actuation element(s) 117'. In some aspects, fastener 139 may be integrally formed with distal membrane 138 or may be a separate component attached to the proximal surface of distal membrane 138. This arrangement may allow for easier assembly and disassembly of the device, as the actuation elements could be readily engaged or disengaged from fastener 139.

In other aspects, positive or negative pressure may be used to move distal membrane 136 proximally or distally. This configuration may allow for actuation of the end effector without the need for separate actuation element(s) 117' proximal of distal membrane 136, although actuation element(s) 117 may be distal of distal membrane 136. In such aspects, upon assembly of second portion 103 with first portion 101, cover 120 may create a fluid-tight barrier around shaft 104. Handle 102 may be configured to fluidly connect to a fluid source. Positive or negative pressure may be applied to the lumen of shaft 104 to control the movement of distal membrane 136. For example, when positive pressure is applied to the lumen of shaft 104, distal membrane 136 may be pushed distally. This distal movement of the membrane may result in actuation or activation of jaws 124. Conversely, when negative pressure is applied, distal membrane 136 may be pulled proximally, potentially causing a different actuation or deactivation of jaws 124.

In some aspects, the pressure-based actuation system may provide a simplified mechanism for controlling end effector 108, reducing the number of components and potentially improving reliability. The fluid-tight seal created by cover 120 may allow for precise control of the pressure within the lumen of shaft 104, enabling fine adjustments to the position of distal membrane 136 and corresponding movements of end effector 108.

In alternative embodiments, cover 120 may be configured without distal membrane 136, thereby featuring a distal opening at its end. In such configurations, the distal opening 130 of base portion 128 effectively serves as the terminal aperture of cover 120, allowing direct access between the interior of shaft 104 and end effector 108 while maintaining the external protective barrier function of cover 120 along the length of shaft 104, as discussed above.

Shown more clearly in FIG. 2B, distal portion 103 may include one or more rings 132 that secure cover 120 to base portion 128. Rings 132 may have an outer diameter that closely matches the inner diameter of base portion 128, so that cover 120 is squeezed securely between rings 132 and base portion 128. Rings 132 may be configured to interact with complementary indentations disposed on an outer surface of shaft 104. In some aspects, rings 132 interact with the coils of shaft 104 (rings 132 sit in the valleys between coils) such that an end effector assembly having end effector 108 is removably coupled to shaft 104 via a threaded or snap fit. In other aspects, rings 132 may include tabs or protrusions that interact with the features (e.g., coils or indentations) of shaft 104 such that end effector 108 is removably coupled to shaft 104 via a snap fit. The tabs or protrusions may extend radially inward from an internal surface of base portion 128.

A distal end of shaft cover 120 may be fixed to end effector 108 in various other ways. For example, a distal end of shaft cover 120 may be fixed to internal surface 134 of base portion 128 via an adhesive or epoxy.

In some aspects, shaft 104 and the inner surface of base portion 128 may have suitable matching diameters or other features such that end effector 108 is removably coupled to shaft 104 via a friction fit. For example, as shown in FIG. 2B, base portion 128 may include at least one slit 138 (for example, 4 slits spaced equally about a circumference of base portion 128) extending proximally from a distalmost end of base portion 128 or distally from a proximalmost end of base portion 128 (as shown in FIG. 2B). Described in further detail below, slit 138 may be configured to permit base portion 128 to expand, for example, upon receiving the distal end of shaft 104. Additionally or alternatively, slit 138 may enable base portion 128 to expand to assist with the removal of end effector 108 from shaft 104, for example, upon disassembly or removal of end effector 108 from the distal end of shaft 104. In some aspects, slit 138 may enable base portion 128 to expand or contract, for example, to accommodate various tolerances associated with shaft 104 and/or base portion 128.

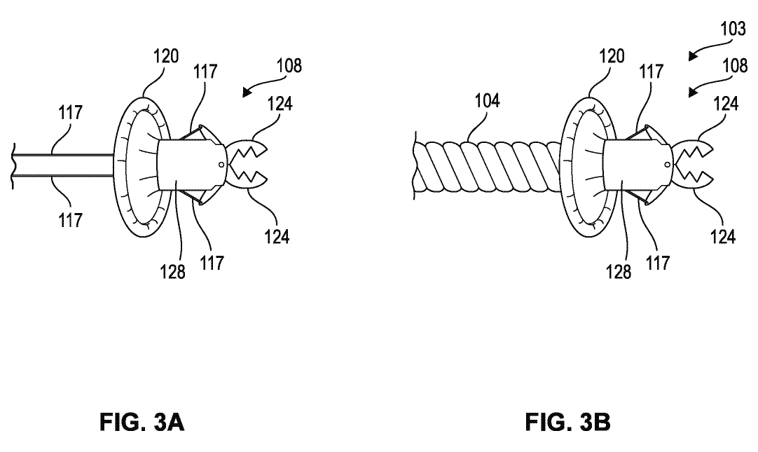

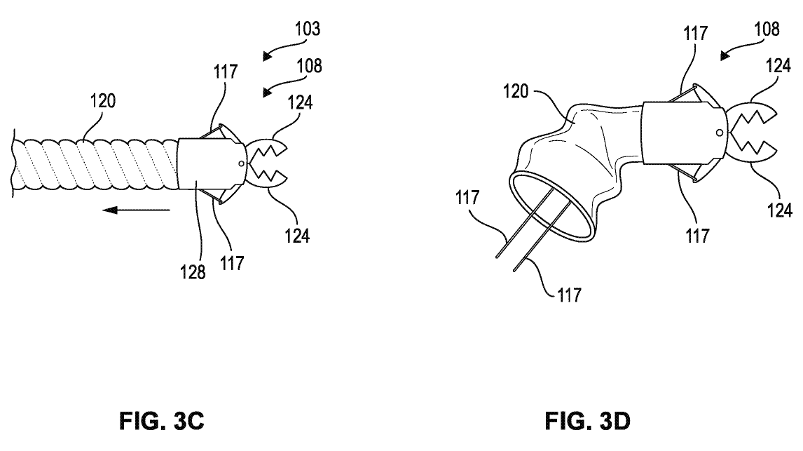

FIGS. 3A-3D illustrate end effector 108 in various states of assembly. For example, FIG. 3A illustrates end effector 108 in a first configuration (e.g., prior to being assembled with shaft 104). In these aspects, end effector 108 may be provided separately from handle 102 and shaft 104. Shaft cover 120 may be in a rolled or partially rolled configuration prior to being assembled. Actuation element(s) 117 may extend proximally from a proximal end of the jaws 124, through opening 130 of base portion 128 and through distal membrane 136 of shaft cover 120. Jaws 124 may be in an open or closed configuration.

FIG. 3B illustrates end effector 108 in a second configuration (e.g., in a first assembly step). In this configuration, actuation element(s) 117 are fed through lumen of shaft 104 and proximally to handle 102. A proximal end of actuation element(s) 117 may be coupled directly or indirectly to one or more actuators 109 of handle 102. The distal end of shaft 104 is fixed to base portion 128, thus securing end effector 108 to shaft 104. In the second configuration, shaft cover 120 may still be in the rolled or partially rolled configuration.

FIG. 3C illustrates end effector 108 in a third configuration (e.g., in a second assembly step). In this configuration, shaft cover 120 is unrolled proximally and secured to a proximal portion of shaft 104 or to handle 102, as described in further detail below. In this configuration, shaft cover 120 may fit around shaft 104, similar to a flexible sheath. In aspects, shaft cover 120 may expand or stretch to conform to shaft 104 and fit snugly around shaft 104.

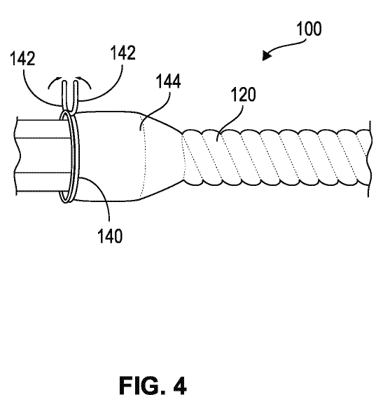

For example, FIG. 4 illustrates a side view of a portion of medical device 100 and illustrates an exemplary configuration for securing a proximal end of shaft cover 120 to first portion 101 of medical device 100. As described above, the proximal end of shaft cover 120 may be secured to handle 102, or to a proximal portion of shaft 104. In embodiments, shaft cover 120 is secured to medical device 100 such that the proximal end of shaft cover 120 is outside of the subject.

Shaft cover 120 may be secured to medical device 100 using a clamp 140. Clamp 140 may be configured to surround shaft cover 120 and a portion of handle 102. Additionally or alternatively, clamp 140 may be configured to surround shaft cover 120 and a portion of shaft 104. In aspects, clamp 140 may include a pair of lever arms 142. With the diameter of clamp 140 expanded, clamp 140 may be positioned onto medical device 100. Upon releasing lever arms 142, the inner diameter of clamp 140 may decrease to conform to medical device 100. Accordingly, shaft cover 120 may be positioned and secured between clamp 140 and medical device 100.

In some aspects, shaft cover 120 may include an expanded proximal portion 144 to conform to the contours of handle 102. Accordingly, expanded proximal portion 144 may facilitate application of shaft cover 120 to medical device 100. Shaft cover 120 may include markings along all or parts of the longitudinal length of shaft cover 120. The markings may assist a user in determining a position of end effector 108 within the subject’s body.

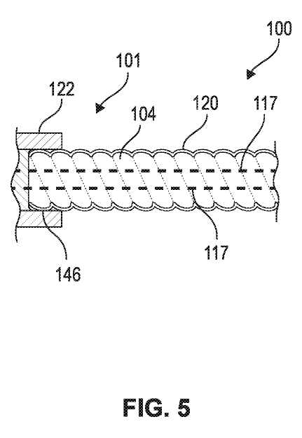

FIG. 5 illustrates a cross-sectional view of a portion of medical device 100 and illustrates an alternative exemplary configuration for securing a proximal end of shaft cover 120 to first portion 101 of medical device 100. For example, as discussed above with respect to FIG. 1, handle 102 may include a distal lock 122. Distal lock 122 may be configured to secure a proximal end of shaft cover 120 between a proximal end of shaft 104 and distal lock 122. An internal surface 146 of distal lock 122 may include locking features (e.g., threads, protrusions, indentations, etc.) configured to interact with shaft 104. Such features may be similar to those described above with respect to base portion 128 of end effector 108. Distal lock 122, once attached to shaft 104 and shaft cover 120, may be coupled to another portion of handle 102, for example, by screwing distal lock 122 onto that other portion of handle 102.

Referring back to FIG. 3C, in the third configuration, medical device 100 may be used. During use, end effector 108 and a portion of shaft 104 is inserted into the subject (e.g., via a natural orifice, incision, or working channel of another device). Shaft cover 120 may provide a barrier between the reusable shaft 104 and the subject. A user may use multiple end effectors 108 one at a time, including multiple different end effectors 108, with a same shaft 104 throughout a single procedure by removing end effector 108 from shaft and attaching a new end effector 108 to shaft 104, as described below.

FIG. 3D illustrates end effector 108 removed from shaft 104. For example, to remove end effector 108, the proximal ends of actuation element(s) 117 may be de-coupled or detached from actuator(s) 109. The proximal end of shaft cover 120 may also be de-coupled or detached from the proximal portion of medical device 100. Shaft cover 120 may be removed by pulling or peeling the proximal end of shaft cover 120 distally over shaft 104. In other aspects, shaft cover 120 may be rolled distally and, accordingly, may turn inside-out.

After removing shaft cover 120 from shaft 104, the distal end of shaft 104 may be released/uncoupled from base portion 128, thereby de-coupling end effector 108 from shaft 104. End effector 108, including base portion 128, may be removed from shaft 104 by pulling shaft 104 and base portion 128 with sufficient force to overcome the snap fit or friction fit. In other aspects, rotating end effector 108 relative to shaft 104 will unscrew and detached end effector 108 from shaft 104.

Upon removing end effector 108 from shaft 104, first portion 101 of medical device 100 may be sterilized and re-used while end effector 108 may be discarded after a single use.

While principles of this disclosure are described herein with the reference to illustrative examples for particular applications, it should be understood that the disclosure is not limited thereto. Those having ordinary skill in the art and access to the teachings provided herein will recognize additional modifications, applications, and substitution of equivalents all fall within the scope of the examples described herein. Accordingly, the invention is not to be considered as limited by the foregoing description.

We claim:

1. A medical device, comprising:
a handle;
a shaft extending distally from the handle;
an end effector assembly removably coupled to a distal end of the shaft; and
a shaft cover fixed to the end effector assembly, wherein the shaft cover is configured to extend proximally over at least a portion of the shaft, wherein, in a first configuration of the shaft cover, the shaft cover is at least partially rolled.

2. The medical device of claim 1, wherein, in a second configuration of the shaft cover, the shaft cover is unrolled and extends proximally along at least a portion of a longitudinal length of the shaft.

3. The medical device of claim 2, wherein, in the second configuration, the shaft cover extends along an entire longitudinal length of the shaft.

4. The medical device of claim 1, wherein the shaft cover includes a membrane extending across an opening at a proximal portion of the end effector assembly.

5. The medical device of claim 4, further comprising an actuation element extending proximally from the end effector assembly, wherein the actuation element extends through the membrane.

6. The medical device of claim 4, further comprising:
a first actuation element extending from an end effector of the end effector assembly and terminating at a distal side of the membrane; and
a second actuation element extending from the handle and terminating at a proximal side of the membrane,
wherein the membrane is disposed between the first actuation element and the second actuation element.

7. The medical device of claim 6, wherein a distal end of the second actuation element is coupled to a fastener disposed on the membrane.

8. The medical device of claim 7, wherein the fastener is integrally formed with the membrane.

9. The medical device of claim 1, wherein the end effector assembly includes a base portion configured to receive a distal end of the shaft, wherein the base portion defines an opening through which an actuation element extends.

10. The medical device of claim 9, wherein the base portion includes one or more protrusions extending radially inward from an internal surface of the base portion, wherein the one or more protrusions are configured to interact with complementary indentations disposed on an outer surface of the shaft.

11. The medical device of claim 9, wherein the base portion includes a slit extending distally from a proximal end of the base portion, and wherein the base portion is configured to at least partially expand at the slit upon receiving the distal end of the shaft.

12. The medical device of claim 1, wherein a proximal end of the shaft cover is secured to the handle via a clamp configured to surround the shaft cover and a portion of the handle.

13. The medical device of claim 1, wherein the handle includes a distal lock configured to secure a proximal end of the shaft cover between a proximal end of the shaft and the distal lock.

14. The medical device of claim 1, wherein the end effector assembly includes an end effector having a pair of jaws movably fixed relative to one another, and a pair of arms extending distally from a base portion.

15. The medical device of claim 1, wherein the shaft includes a flexible coil.

16. A medical device, comprising:
a handle;
a shaft extending distally from the handle;
an end effector assembly removably coupled to a distal end of the shaft, wherein the end effector assembly includes an end effector and a base portion defining an opening; and
a shaft cover fixed to the end effector assembly, the shaft cover including a membrane extending across the opening, wherein the membrane is a barrier to inhibit material from entering a lumen of the shaft.

17. The medical device of claim 16, further comprising an actuation element extending proximally from the end effector, wherein the actuation element extends through the membrane and extends proximally through the shaft to the handle.

18. The medical device of claim 16, further comprising:
a first actuation element extending proximally from the end effector and terminating distally of the membrane; and
a second actuation element extending from the handle and terminating proximally of the membrane, wherein the membrane is between a proximalmost end of the first actuation element and a distalmost end of the second actuation element.

19. The medical device of claim 18, wherein the distalmost end of the second actuation element is secured to a fastener disposed on a surface of the membrane.

20. A distal end effector assembly of a medical device, comprising:
a pair of jaws movably fixed relative to one another;
an actuation element extending proximally from a proximal end of the jaws;
a base portion, wherein a proximal end of the base portion is configured to removably receive a distal end of a shaft, wherein the base portion includes an opening through which the actuation element extends; and
a shaft cover fixed to the base portion, wherein the shaft cover includes a membrane extending across the opening, wherein the membrane is configured as a barrier to prevent bodily fluids, debris, or contaminants from entering a lumen of the shaft.
